(19) United States
(12) Patent Application Publication
(10) Pub. No.: US 2006/0229545 A1
(43) Pub. Date: Oct. 12, 2006

(54) Method of minimizing wrinkles

(76) Inventor: James Zaguroli JR., Drayton Plains, MI (US)

Correspondence Address: JOHN R. BENEFIEL, 280 DAINES ST., #100 B, BIRMINGHAM, MI, 48009, US

(21) Appl. No.: 11/399,618

(22) Filed: Apr. 6, 2006

Related U.S. Application Data
(60) Provisional application No. 60/669,074, filed on Apr. 6, 2005.

(51) Int. Cl. A61B 19/00

(52) U.S. Cl. 602/58

(57) ABSTRACT

A method of minimizing wrinkles in the human skin involves stretching out the skin adjacent a wrinkle to flatten the wrinkle and applying a three layer piece onto the stretched adjacent skin and over the wrinkle while holding the skin taut to flatten out the wrinkle. An adhesive inner layer holds the tape on the skin, while an intermediate stiffener layer holds the wrinkle and skin in the stretched out condition. An outer breathable layer covers the intermediate layer and an outer perimeter of the inner layer which cushions a perimeter edge of the intermediate layer and prevents forming lines in the skin by being held in tight contact with the intermediate layer over an extended period of time.

### CROSS REFERENCE TO RELATED PATENT APPLICATIONS

This application claims the benefit of U.S. provisional application 60/669,074 filed on Apr. 6, 2005.

### BACKGROUND OF THE INVENTION

This invention concern methods for minimizing wrinkles in the facial skin of a human.

Winkles have long been though to be undesirable since they are a sign of aging and are unattractive in appearance.

Methods and devices have heretofore been developed in an attempt to minimize skin wrinkles in humans, typically involving mud packs, lotions, etc. Surgical procedures such as “face lifts” have also been developed, and the use of botox injections have recently been employed to minimize wrinkles. These treatments are typically burdensome, inconvenient, or expensive.

One simple technique previously devised has been to apply a flexible piece of tape to the skin while holding taut the skin area adjacent a wrinkle to be treated.

A product sold for many years under the trademark “FROWNIES” utilizes a paper sheet and an adhesive gum to make the tape used in this treatment.

U.S. Pat. No. 3,949,741 describes a similar technique using an adhesive coated flexible plastic film.

While this technique is of some limited effectiveness, the present invention has as an object an enhancement thereof to be more effective at minimizing wrinkles.

### SUMMARY OF THE INVENTION

The above object and other objects which will become apparent upon a reading of the following specification and claims, are achieved by applying a wrinkle treatment multilayered tape piece across a skin wrinkle, while the adjacent skin areas is held taut to flatten out the wrinkle. The tape piece includes an outer flexible breathable layer of woven material or a plastic film layer. An intermediate flexible plastic stiffener layer is held between the outer layer and a double adhesive cushioning inner layer which may be of foam or woven material. The tape piece is releasably held on a transfer sheet prior to being applied to a user's skin. The stiffener layer is completely surrounded by the outer layer and the cushioning layer so that the perimeter edge of the relatively stiff layer is cushioned entirely around its perimeter to prevent the formation of lines in the skin of the user by the stiffener layer being held tightly against the skin for an extended period.

The inclusion of the stiffener layer creates a greater stretch holding power on the skin so as to enhance the wrinkle mitigating effect over the simple flexible tape strips heretofore used in treating wrinkles.

### DESCRIPTION OF THE DRAWINGS

FIG. 1 is pictorial view of a skin treatment tape piece utilized in the method according to the invention.

FIG. 2 is an exploded pictorial view of the several tape piece layers.

FIG. 3 is a pictorial view of a treatment tape piece applied to a patient's face.

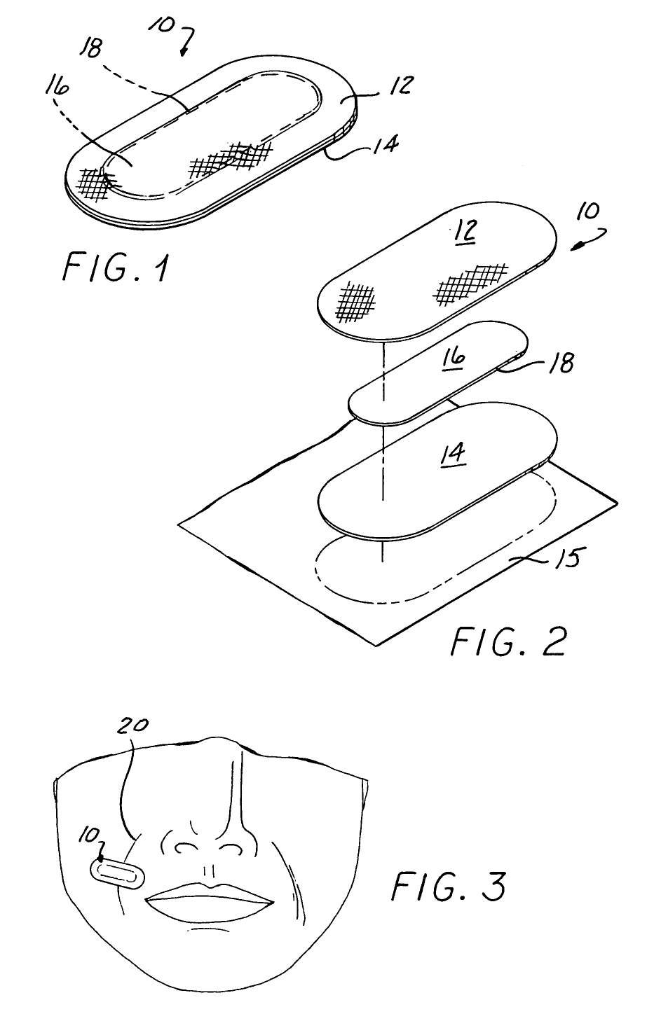

### DETAILED DESCRIPTION

In the following detailed description, certain specific terminology will be employed for the sake of clarity and a particular embodiment described in accordance with the requirements of 35 USC 112, but it is to be understood that the same is not intended to be limiting and should not be so construed inasmuch as the invention is capable of taking many forms and variations within the scope of the appended claims.

Referring to the drawings, the method according to the invention involves the use of a multilayered wrinkle treatment tape piece 10.

The multilayer construction of the tape piece 10 is similar to that used in a nasal “dilator” device sold under the trademark BREATH RIGHT, and described in U.S. Pat. Nos. 5,533,503 and 6,318,362 incorporated by reference herein.

An outer cover layer 12 is formed of a breathable and compliant woven or plastic film material.

A stiff but resiliently bendable intermediate layer 16 of thin plastic sheet is secured beneath the outer cover layer 12 which is smaller in area than the cover layer 12, having a shorter length and width so as to be completely surrounded by the cover layer 12 as well as by a double sided adhesive cushioning inner layer 14 which is of the same configuration as the outer cover layer 12 and aligned therewith.

A transfer sheet 15 holds the treatment tape piece 10 to be readily releasable until it is placed in use.

The intermediate layer 16 may be of a thin but stiff layer sheet of plastic, i.e. such as polypropylene sheet approximately 0.012 inches thick.

The double backed adhesive cushioning layer may be of foam or woven material layer, and is thick and soft enough to provide a cushioning effect, i.e. on the order of 0.008 inches thick. This prevents the perimeter edge 18 of the intermediate layer 16 from leaving marks on the skin as a result of being held tightly against the skin for extended periods.

The method involves similar steps as described in the prior techniques, i.e. the user stretches the skin with his or her fingers so as to be drawn taut on either side of a wrinkle 20 so as to flatten out the wrinkle 20.

The treatment tape piece 10 is removed from the transfer sheet 15 and applied to the skin across the wrinkle 20 while holding the skin taut.

The tape piece is left in position for several hours, preferably overnight. This is repeated daily as necessary.

It has been found that the added stiffness afforded by the intermediate stiffener layer 16 holds the skin taut to produce a more effective minimization of the wrinkle while avoiding leaving marks on the skin after the tape piece is removed.

1. A method of reducing wrinkles in the human skin comprising stretching the skin adjacent the wrinkle to flatten out the wrinkle and applying a multilayer tape piece over the flattened out wrinkle and adjacent stretched skin comprised of an outer flexible cover layer, an intermediate stiffener layer of a stiff but resiliently bendable material able to hold the skin in the stretched out condition, and an inner adhesive layer to hold the piece against the stretched out condition;
leaving said tape piece applied to the stretched skin for an extended period of hours; and, thereafter removing said tape piece.

2. The method according to claim 1 wherein a cushioning adhesive layer is used for the inner layer of said tape piece.

3. The method according to claim 2 wherein said intermediate layer is made to be smaller than said inner layer to create a cushioned perimeter around a perimeter edge of said intermediate stiffener layer.

4. The method according to claim 3 wherein the outer layer is also made larger than said intermediate layer so as to enclose said perimeter of said intermediate layer.

5. The method according to claim 1 wherein said method is repeated daily for a period of days.

6. The method according to claim 2 wherein said inner layer is made of an adhesive foam material.

7. The method according to claim 1 wherein said flexible outer layer is made of a breathable woven material.

8. The method according to claim 1 wherein said intermediate stiffener layer is made of a thin plastic sheet material.
